(19) United States
(12) Patent Application Publication
(10) Pub. No.: US 2025/0025307 A1
(43) Pub. Date: Jan. 23, 2025

(54) Custom Bone Scaffold Using Tessellation of Polygons

(71) Applicant: Boneforger Holding Company, Dover, DE (US)

(72) Inventors: Jacob Millburn, Dover, DE (US); Kirk Zu Jiao, Dover, DE (US)

(73) Assignee: Boneforger Holding Company, Dover, DE (US)

(21) Appl. No.: 18/424,811

(22) Filed: Jan. 28, 2024

Related U.S. Application Data
(60) Provisional application No. 63/514,347, filed on Jul. 18, 2023.

(51) Int. Cl. A61F 2/30; A61F 2/28

(52) U.S. Cl. CPC A61F 2/30771; A61F 2/28; A61F 2/30942; A61F 2002/30062; A61F 2002/30113; A61F 2002/30143; A61F 2002/3092; A61F 2002/30985

(57) ABSTRACT

A bone scaffold structure may include one or more scaffold units, wherein each of the scaffold units comprises a first end profile, a second end profile, and one or more wires connecting the first end profile to the second end profile. Each of the first end profile and the second end profile may be defined by a polygon surface that is specified by a center and a number of vertices that are pairwise connected by a side. Each of the one or more wires may be specified by a wire length and a substantially circular cross-section with a first end of each of the one or more wires being connected to a vertex of the polygon surface defining the first end profile and a second end of each of the wires being connected to a vertex of the polygon surface defining the second end profile.

### CROSS-REFERENCE TO RELATED APPLICATIONS

This application claims priority benefit from U.S. Provisional Application No. 63/514,347 filed Jul. 18, 2023, the content of which is incorporated by reference herein.

### TECHNICAL FIELD

The present disclosure relates generally to custom bone scaffold structures. More specifically the disclosure relates to bone scaffold structures that can be easily manufactured to specifications based on an integral combination of polygonal shaped units.

### BACKGROUND

Many patients may suffer from bone injuries resulting in bone fractures. These injuries may be caused by accidents (e.g., sports-related injuries) or by diseases (e.g., osteoporosis). Osteoporosis is a common condition among aging populations that causes bones to deteriorate, becoming more porous and subsequently more fragile with the increase of a person's age. Many people with osteoporosis may suffer severe fractures caused by a fall due to the reduced bone density. The treatments of these kinds of bone fractures may be uniquely difficult and expensive.

Current bone fracture repairs may include external and/or internal fixations. For simple fracture repair, a cast is used as an external fixator and the fracture may self-regenerate. For more difficult fracture repair, expensive metal or metalloid hardware is installed in the fracture site to affix the bone fragments in place during the healing process. However, these implants commonly require removal in a second surgery to prevent long term issues ranging from ion leaching to trouble with security checkpoints and metal detectors, including MRI machines. Furthermore, many patients do not undergo the second surgery to have the implants removed post-healing. Still further, these fixation methods often require additional hardware, including but not limited to screws, pins, bolts, clips, sutures, clamps, and/or wrappings.

Many current implant designs are not optimized according to a person's physical and biological attributes. For example, bone may need to receive mechanical load in order to grow, but current implant technologies are focused on implant survivability and durability rather than long term repair successes. These implants are also typically solid, which may interfere with revascularization of the bone if the implant is not removed in a timely manner.

### BRIEF DESCRIPTION OF THE DRAWINGS

In the drawings, which are not necessarily drawn to scale, like numerals may describe similar components in different views. Like numerals having different letter suffixes may represent different instances of similar components. Some implementations are illustrated by way of example, and not limitation, in the figures of the accompanying drawings.

FIG. 1 illustrates a hexagon surface of an end profile of a scaffold unit, according to some implementations of the present disclosure.

FIG. 2 shows a section of a tessellation of scaffold unit end profiles that are each defined by a hexagon surface, according to some implementations of the present disclosure.

FIG. 3 shows a hexagonal profile outline, for a bone scaffold structure, selected from a tessellation of hexagons, according to some implementations of the present disclosure.

FIGS. 4A-4C show views of a bone scaffold structure and profile configurations based on a tessellation of hexagon surfaces, according to implementations of the present disclosure.

FIGS. 5A-5B show views of a cage structure of a scaffold unit and a cross section of the cage structure of the scaffold unit, according to some implementations of the present disclosure.

FIGS. 6A-6B show views of a scaffold structure with end profiles rotated to different angles around a central axis, according to implementations of the present disclosure.

FIG. 7 shows a flowchart for a method for manufacturing a bone scaffold structure, according to some implementations of the present disclosure.

FIG. 8 is a block diagram illustrating a machine in the example form of a computer system, within which a set or sequence of instructions may be executed to cause the machine to perform any one of the methodologies discussed herein, according to some implementations of the present disclosure.

### DETAILED DESCRIPTION

Current treatments for bone fractures may include bone implants (e.g., hip implants used for treating osteoporotic hip fracture in the elderly) or revision surgeries to treat dislocation and infection. In this disclosure, a bone implant refers to a surgical procedure that employs natural or artificial scaffolds for osseous reconstruction that may repair bone fractures. A scaffold is a three-dimensional physical structure or framework that allows and stimulates the attachment and proliferation of osteo-inducible cells or tissues on its surfaces. In the context of bone implants for treating fractures, these cells or tissues held on a scaffold may help regenerate bones, thus repairing the bone fractures. The natural scaffolds refer to bone grafts from other parts of the same patient (autograft) or from other persons (allograft) and the artificial scaffolds refer to structures or frameworks made from synthetic materials, potentially using biomimetic processes. This disclosure is concerned with the artificial scaffolds and their design and construction. For the conciseness of description, a scaffold structure in this disclosure refers to an artificial scaffold made from synthetic materials hereafter.

One implementation of the disclosure may include a bone scaffold structure including a first profile, a second profile, and wires connecting from connection locations on the first profile to connection locations on the second profile. The connection locations on the first profile may be located at vertices associated with a tessellation of polygonal units on a surface of the first profile, and the connection locations on the second profile may be located at vertices associated with a tessellation of polygonal units on a surface of the second profile.

In one implementation, the tessellation of polygonal units (e.g., hexagons) on the surface of the first profile or the second profile may include identical polygons/hexagons that are adjacently arranged so that a polygon/hexagon shares a common side and two vertices with each one of neighboring polygons/hexagons. Thus, the adjacently arranged polygons/hexagons in the tessellations on the first profile and the second profile may be tightly connected but do not overlap with each other. As to the tessellation of hexagons, each hexagon may be connected to up to six neighboring hexagons.

In an implementation, each of the polygonal units (e.g., scaffold units of the scaffold structure) may be a hexagonal unit that has a structure including two end profiles having a certain thickness and a hexagon surface, where the hexagon surface is specified by a center and six vertices. The hexagonal unit may further include one or more wires that connect from a first end profile to a second end profile of the hexagonal unit. The one or more wires may include a number of wires (e.g., 6) that may have equal lengths, and at first end points connect to the vertices of the hexagon surface of the first end profile and at second end points connect to the vertices of the hexagon surface of the second end profile. In this way, a bone scaffold structure may be defined and designed according to a tessellation of hexagonal units (e.g., scaffold units), therefore allowing a flexible design and construction of a bone scaffold structure for bone implant based on a patient's specific biological measurements.

Implementations of the disclosure may provide a computer-implemented method for designing and manufacturing a bone scaffold structure. The method may include obtaining one or more biological measurements from a human subject. The biological measurements may include, but are not limited to, a bodyweight, a specific bone to be implanted with the bone scaffold structure, a fluid flow rate, an age, and a medical history of the human subject, where the fluid may include, for example, the blood. The method may further include calculating, based on the one or more biological measurements, one or more target physical characteristic parameters associated with the bone scaffold structure. The one or more target physical characteristic parameters may include, but are not limited to, a permeability to fluid flow and a vertical stress sustainability. The method may further include calculating, based on the one or more target physical characteristic parameters, one or more design parameters for manufacturing the bone scaffold structure. The one or more design parameters may include, but are not limited to, a selection of a material of the bone scaffold structure, a number of scaffold units (e.g., polygonal units) required for the bone scaffold structure, a radius and length of the wires used in the scaffold units, a number of wires in each of the one of more scaffold units, and a length of the side that pairwise connects two vertices of the polygon surface defining one of the first end profile or the second end profile of each of the one or more scaffold units.

The method may further include providing the one or more design parameters to a machine to manufacture the bone scaffold structure based on the one or more design parameters. In one implementation, the machine can be a 3D printer that may employ an additive process to construct the bone scaffold structure. The additive process may deposit material layer by layer to quickly construct the 3D form of the bone scaffold structure. The additive process may instead selectively sinter material layer by layer as well. The 3D prototype may be further post-processed to arrive at the bone scaffold structure that is suitable for implant in the patient. The post-processing may include trimming the outer boundaries of bone scaffold structure to fit the patient's needs, polishing and coating the outer surface, sintering, etc. Example materials used to construct scaffolds may include, for example, biometals (e.g., Zimmer Biomet's Trabecular Metal (TM)) and other materials such as bioceramics and resorbable polymers.

To achieve the flexible and personalized design and manufacture of the bone scaffold structure customized to the particular situation of a patient, in one implementation, the bone scaffold structure may be designed to be composed of one or more identical scaffold units. In this way, each identical scaffold unit may be defined by parameterized equations so that its dimensions can be specified and adjusted to accommodate patient needs.

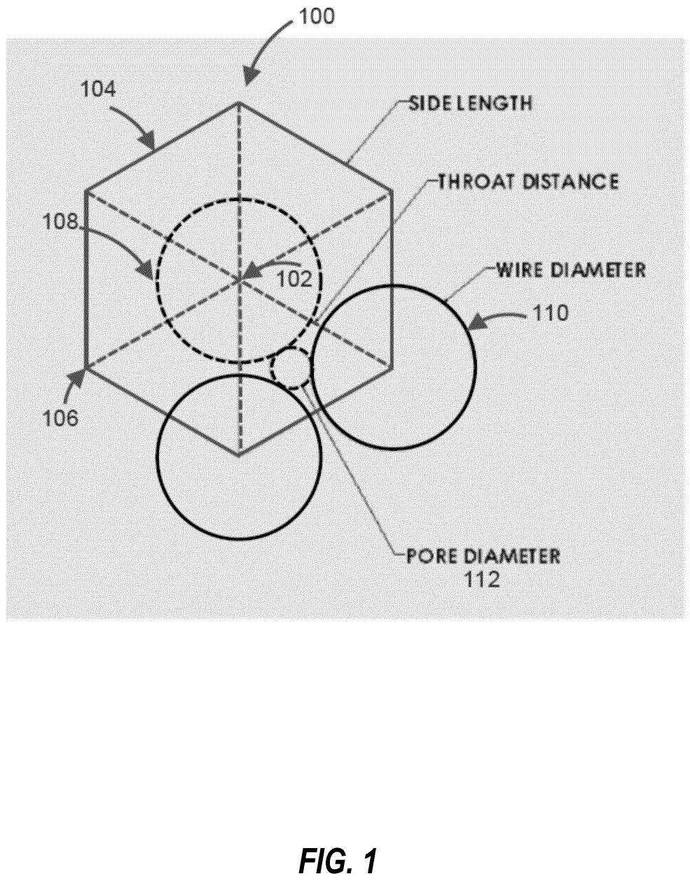

FIG. 1 illustrates a hexagon surface 100 of an end profile of a scaffold unit, according to some implementations of the present disclosure. A scaffold structure can be designed and constructed using scaffold units as the basic building components. Referring to FIG. 1, the scaffold unit may include two end profiles each with a hexagon surface 100. Hexagon surface 100 may be specified by a center 102, six equal-length sides 104, and six vertices 106. The center 102 and the six sides 104 may form six equilateral triangles. Each vertex 106 may be connected to a wire 110 having a circular cross-section. Similarly, the center 102 may be connected to a wire 110 or cut out as a hole 108. The distance between the circumference of a wire 110 connected to a vertex and the circumference of the hole 108 (or of a wire 110) located at the center 102 may be referred to as a “throat distance”, and the space among two adjacent wires 110 and the center hole 108 may be referred to as a “pore” having a pore diameter 112. In this way blood may flow axially through the pores with the pore diameter 112, transversely through the throat distance. Therefore, the choice of a diameter of a wire 110 may be used to determine a fluid flow rate that is optimal for a recovery speed of a patient, where the fluid flow rate is related to the size and quantity of the pores.

Accordingly, a scaffold unit may be specified using a parametric hexagon (e.g., hexagon surface 100) and its component equilateral triangles as shown in FIG. 1. Circles representing wires 110 may be placed at two or three points of one triangle (e.g., vertices). Therefore, the throat distance mentioned above may also be determined as a difference between the side length of a triangle and the diameter of a wire 110 since this may also represent the minimum distance between adjacent wires 110. Cells larger than the throat distance will not be able to pass through the three-dimensional diamond-shaped pores (discussed further below) that are created around an axis of rotation at the center 102 when the wires 110 are extruded. The largest circular area between adjacent wires 110 and/or holes (e.g., pore diameter 112) represents an approximate clearance that may be able to easily vascularize the inner regions of a scaffold unit, as shown in FIG. 1. The scaffold units may be integrally combined together to form a scaffold structure as described more fully below.

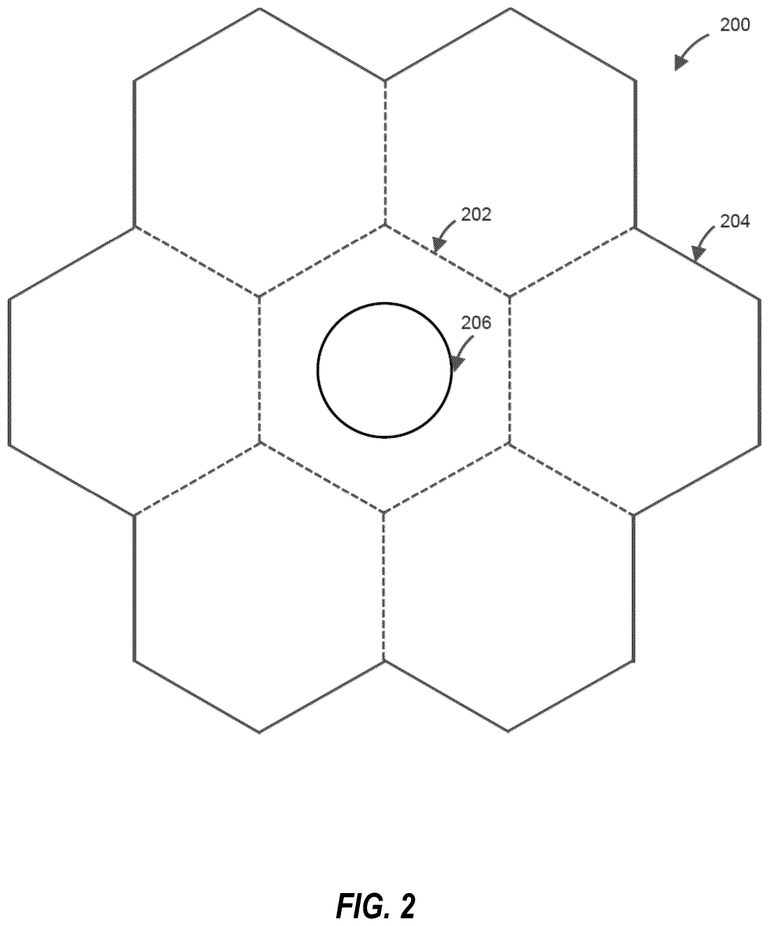

FIG. 2 shows a section of a tessellation 200 of scaffold unit end profiles that are each defined by a hexagon surface 202 or 204, according to some implementations of the present disclosure. In designing a bone scaffold structure for implant, a scaffold unit may be expanded into a tessellation pattern of identical scaffold units. The number and combination of scaffold units may be designed and customized to a patient's physical and biological attributes. Referring to FIG. 2, a section 200 of the tessellation pattern is shown to include a central hexagon surface 202 (e.g., of an end profile of a central scaffold unit) and six adjacent hexagon surfaces 204. Each pair of adjacent hexagon surfaces share a common side without any gap or overlapping. The tessellation of identical hexagon surfaces may be used in order to design an integral bone scaffold structure by using a software application (e.g., SolidWorks®) to copy the identical bone scaffold units defined by the hexagon surface (e.g., 202) as many times as needed (e.g., six copies of 204). For example, the software application may use a fractal expansion algorithm to save modeling time by first designing the scaffold unit and then copying the scaffold unit around itself in a tessellation pattern until the pattern is sufficiently large for the desired profile geometry of the bone scaffold structure (e.g., a sufficient number of bone scaffold units have been produced around the central bone scaffold unit defined by hexagon surface 202). The end product of the bone scaffold structure, however, may be an integral structure without separable scaffold units. In one implementation, the center 206 of the central scaffold unit defined by hexagon surface 202 (e.g., around which the other scaffold units were copied) may be provided with a hole 206 that cuts through the full-thickness of the first end profile and second end profile of the central scaffold unit.

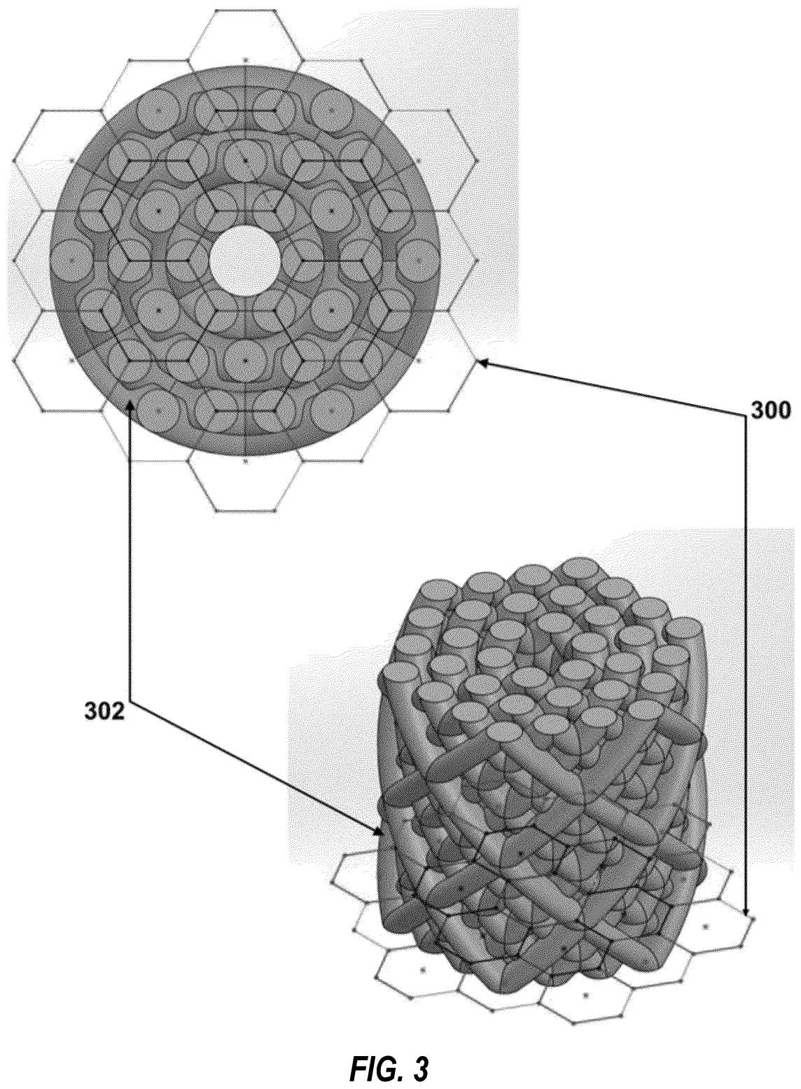

FIG. 3 shows a profile 302, for a bone scaffold structure, selected from a tessellation 300 of end profiles that are each defined by a hexagon surface, according to some implementations of the present disclosure. As noted above, the central hexagon surface of a scaffold unit end profile may be copied into a tessellation pattern 300 (e.g., no gaps or overlapping) around itself until the tessellation pattern is sufficiently large for the desired profile geometry (e.g., profile 302) of a desired bone scaffold structure to be extruded from the tessellation pattern 300. Tessellation 300 may further define the surface shape and area of profile 302 of the scaffold structure. To this end, the profile 302 may have a certain thickness and may have been made from a certain metallic material such as, for example, synthetic materials, manufactured using biomimetic processes.

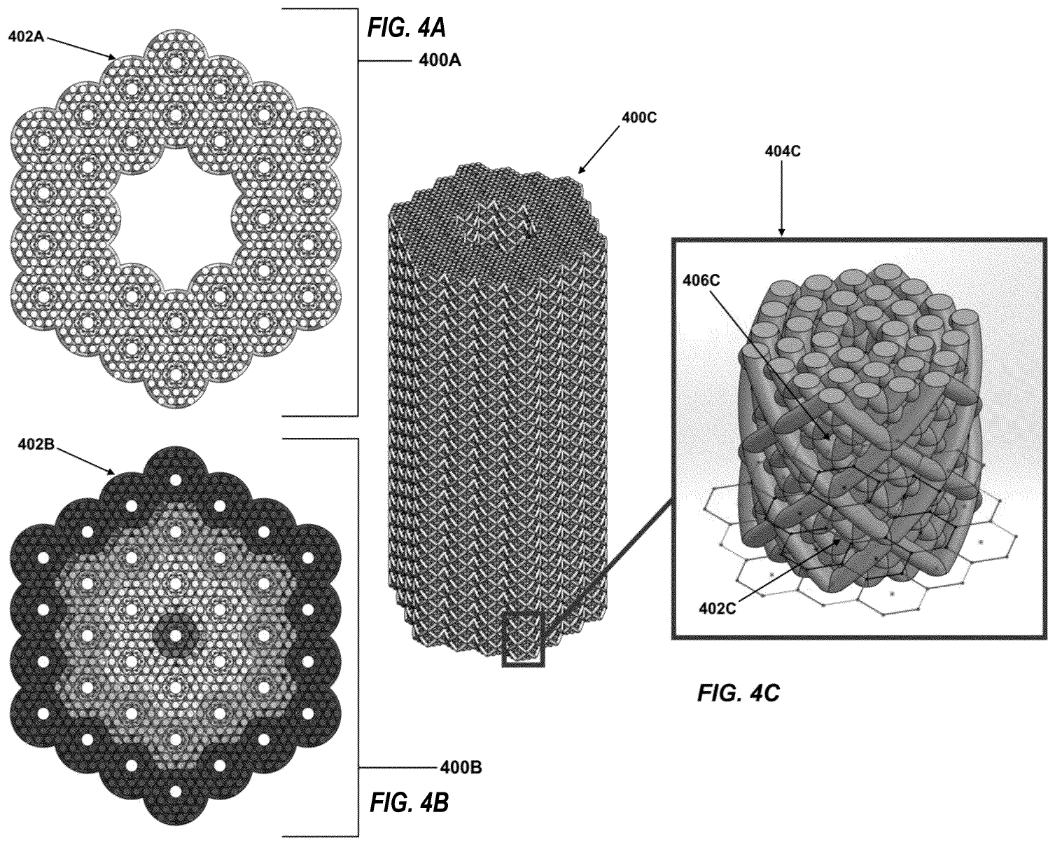

FIGS. 4A-4C show views of a bone scaffold structure 400C and profile configurations 400A and 400B based on a tessellation of hexagon surfaces (e.g., 402A, 402B), according to implementations of the present disclosure.

In one implementation, the bone scaffold structure 400C may be designed so that the centers of specific end profiles 402B of the scaffold units (e.g., at a specific layer of scaffold units in the scaffold structure 400C) may include central holes (e.g., as described above) that may act as the centers of mimicked Haversian canals (e.g., small tubes which form a network in bones and contain blood vessels) for passage of blood/nutrients through the bone scaffold structure 400C. In some implementations, the specific end profiles 402B with central holes may provide wires from the surrounding scaffold units. The specific end profiles 402B may then be rotated so that the wires connected to the specific end profiles 402B may form a cage-like structure 404C around the centers of these specific end profiles 402B with cross-links between the concentric and counter-rotating wires that form diamond-shaped pores 406C to allow for filtration of blood and bone cells.

As shown in each of FIGS. 4A-4C, the fractal expansion/replication pattern for polygons mentioned above may start with a central unit (e.g., scaffold unit of specific end profile 402C), a first radial pattern of identical units around the central unit and a second radial pattern of identical units around the outermost layer, and so on. A scaffold may include compositions of populated and unpopulated layers of scaffold units as in 400A, in which the first unit and first radial pattern have been removed.

Furthermore, there could be a single center of rotation around a central hole through the root (e.g., central) hexagon shaped surface of a specific end profile 402C as shown in FIG. 4C, or there could be numerous centers of rotation around an equal number of central holes, one through the root hexagon surface of a specific end profile 402C and one through the central hexagon surfaces of each of the copies of identical units formed around and adjacent to the central unit in the radial expansion described above as shown respectively in the bone scaffold structure profiles 400A of FIG. 4A and 400B of FIG. 4B. As noted above, these centers of rotation around the central holes through each hexagon surface of a specific end profile 402C may allow nutrient and cell flow through the respective scaffold units and the resulting bone scaffold structure 400C as shown in FIG. 4C.

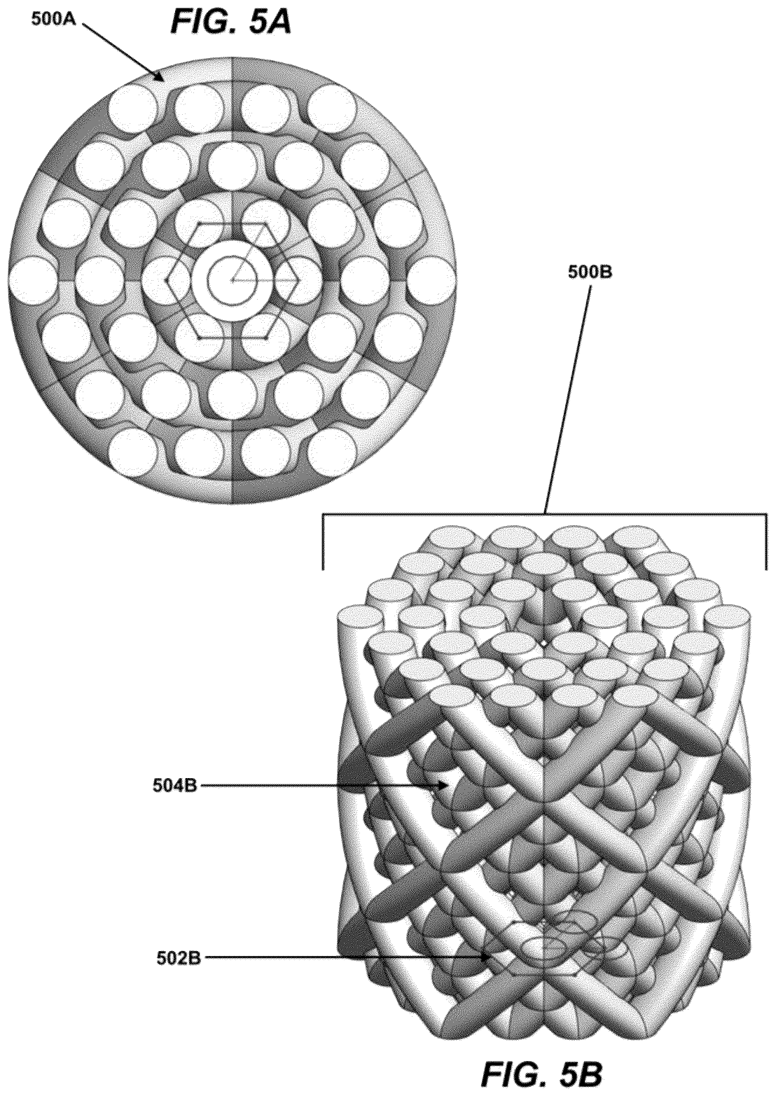

FIGS. 5A-5B show views of a cage structure 500B (e.g., identical to cage structure 404C of FIG. 4C) of a scaffold unit and a cross section 500A of the cage structure 500B of the scaffold unit, according to some implementations of the present disclosure. As shown in FIG. 5B, the scaffold unit may include a hole (e.g., hole 206 of FIG. 2) in the center of a hexagon surface 502B of its end profile (e.g., hexagon surface of FIG. 5A) and may also include diamond-shaped pores 504B that form along the wire walls of cage structure 500B as described above. Controlling the dimensions of these diamond-shaped pores 504B of the cage structure 500B may determine the filtration properties of the scaffold unit and any scaffold structure (e.g., scaffold structure 400C of FIG. 4C) that includes them. Relevant characteristics of this cage structure 500B may include a number of pores and pore regularity, pore dimensions for filtration, and surface and bulk porosities.

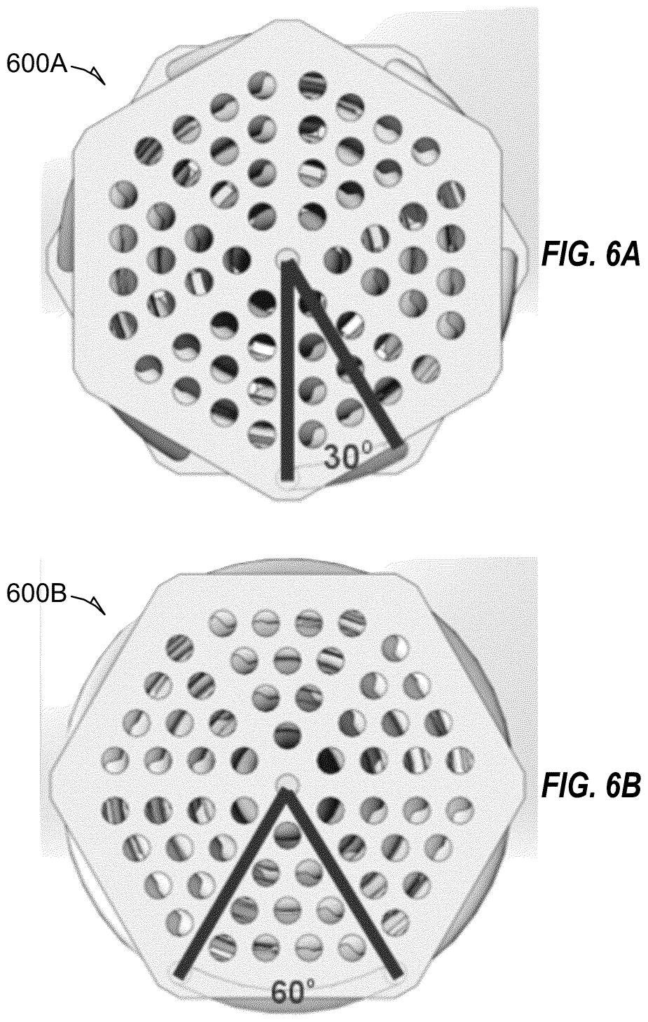

FIGS. 6A-6B show views of scaffold units 600A and 600B with respective end profiles rotated to different angles around a central axis, according to some implementations of the present disclosure. As an initial matter, the pattern of wire connections is discussed. For bilateral opposition of one layer (e.g., one radial pattern around the central hexagon of the cage structure of a scaffold unit), wire connections may be formed for every 30 degrees of radial travel around a central axis of the scaffold unit. For unilateral opposition, however, connections may be formed at every 60 degrees starting at 30 degrees.

For parallel layers, connections may not form between adjacent wires because adjacent wires within that layer are parallel to one another. These layers do not form pores and typically require an endplate in many implementations. Scaffold units including parallel layers can be mechanically controlled by the angle and distance between connection points on the pair of hexagonal surfaces. For example, the end profiles of scaffold unit 600A of FIG. 6A show a 30 degree rotation with respect to the central axis, and the end profiles of scaffold unit 600B FIG. 6B show a 60 degree rotation with respect to the central axis at some predetermined heights.

In both cases, the first connection forms six half-diamond-shaped pores. Thus, the number of diamond-shaped pores created in each opposition, n, can be calculated as follows with respect to the angle of rotation 0 for bilateral (nab) and unilateral (mi) opposition respectively:

$$nb = 6 * ( \frac{θ - 15}{30} )$$

$$nu = 6 * \frac{θ}{60}$$

Because each pore is radial, the representation of each pore may be simplified to a rhombus represented by horizontal and vertical diagonals. The horizontal diagonal is exactly equal to T, the throat distance described above (e.g., the difference between the side length S of the triangle and the diameter W of the wire represents the minimum distance between adjacent wires). Because this rhombus may be divided into four triangles along the diagonals, the vertical distance Vis the product of T and the tangent of the inclination angle φ between the wire and the horizon line. Taking half of the product of T and I yields the area of a diamond-shaped pore as shown below:

T=S−W

V=T*tan(φ)

$$φ = {tan}^{- 1} ( \frac{H}{2 π s * \frac{θ}{360}} )$$

Pore Area=½*V*T

Based on the calculated unit pore area (e.g., of each diamond-shaped pore), the surface and bulk porosities of the cage structure may be determined. The surface porosity may be the ratio of voids (e.g., diamond-shaped pores) to overall footprint of a surface (e.g., cage walls formed by the wires), while the bulk applies to the corresponding volumes. The porosity of the cylindrical cage structure may control the diffusion rate across the scaffold unit, so the cylindrical cage structure may be unwrapped into a rectangle of base length equal to the circumference 2πs and height equal to the height H of the scaffold unit. Each diamond-shaped pore may be represented as a diamond-shaped hole in this rectangle. Thus, the surface porosity (ps) may be determined as the ratio of the total area of the pores to the area of the rectangle:

$$ps = \frac{n * ( \frac{1}{2} VT )}{2 π s * H}$$

Bulk porosity (pb) may be calculated in a similar manner. The overall footprint of the cage structure may be a cylinder of equal height H with a radius equal to the side length S of the hexagon surface of the end profile plus the wire radius or half the wire diameter (e.g., 1/2 W). To find the occupied volume, the volume of each intersection (e.g., cross-link) may be subtracted from the total volume of the wires to avoid double counting. There is a same number of pores as there is of intersections. However, rather than calculating the volume of an intersection, it is simpler to calculate the traversed length of an intersection and subtract the volume of one wire that crosses it. First, width X and height Y of the intersection are calculated using the law of cosines, the intersecting wire diameters W, and the inclination angle q. Next, the transverse distance across the intersection A is calculated using the Pythagorean theorem. Finally, the bulk porosity pb may be calculated from the total volume of wires divided by the total footprint volume of the cage structure as shown below:

$$X = \sqrt{2 {W}^{2} ( 1 - cos ( 180 - 2 φ ) )}$$

$$Y = \sqrt{2 {W}^{2} ( 1 - cos ( 2 φ ) )}$$

$$A = \sqrt{{X}^{2} + {Y}^{2}}$$

$$pb = 1 - \frac{{( \frac{W}{2} )}^{2} ( 12 H - nA )}{{( s + \frac{1}{2} W )}^{2} H}$$

As discussed above, this cage structure may serve as the core filtration mechanism in opposition bone scaffold units designed using this hexagonal tessellation pattern. A computer-implemented method may be executed to calculate scaffold height H, “pore” diameter P, wire diameter W, and pitch angle o as numerical inputs, in addition to deciding on their opposition rules (e.g., bilateral, unilateral, or parallel in certain form of combinations) and pore locations in the cage structure.

For example, as shown in FIG. 5B, the cage structure 500B of a scaffold unit may be modeled to have a scaffold height of 5 mm, a pore diameter of 0.25 mm, a wire diameter of 0.50 mm, and a pitch angle of 150 degrees. Furthermore, this cage structure 500B (like the Haversian model mentioned above) uses bilateral opposition rules.

In order, to evaluate the impact of wire pitch angle on bone scaffold stiffness and examine the dimensional validity of a 3D printing process for manufacturing, a scaffold unit may be modeled based on a few conditions. First, this bone scaffold structure will have only one axis of rotation through the center of the root hexagon surface of the central scaffold unit. Second, each connection point produces one wire, rather than the two that are required to complete the diamond-shaped pores. Both of these conditions work to significantly reduce the number of cross-links formed between wires within the cage structure of the scaffold unit, highlighting the impact of the change in wire pitch angle on the stiffness of the scaffold unit directly rather on than also being based on the number of cross-links formed by the wires.

Three straight-walled scaffold units (e.g., no rotation), three 30-degree rotation central cage scaffolds, and three 60-degree rotation central cage scaffolds were 3D printed (e.g., using a Snapmaker Artisan v2 device) using the parameters outlined in Table 1 below. The resulting 30 degree rotation scaffold unit and the 60 degree rotation scaffold unit correspond respectively to scaffold units 600A of FIG. 6A and 600B of FIG. 6B respectively. These scaffolds were subjected to compressive loading (e.g., in an Instron device) up to 3 kN at a rate of 2 mm/min until either 10% strain or 3 kN.

TABLE 1

| | | |
|---|---|---|
| Pore diameter (PD) | 0.5 | mm |
| Wire diameter (WD) | 2 | mm |
| Throat distance (TD) | ~0.165 | mm |
| Side length (SL) | ~2.165 | mm |
| Dimensioned Height | 10 | mm |

To determine the accuracy and repeatability of the 3D printing process, a cross-section of each scaffold unit was printed at each 2 mm interval along the height of each scaffold unit type (e.g., type based on rotation angle). The sectioned surfaces were stained and imaged (e.g., using ImageJ). ImageJ's particle analysis tools were used to determine the areas of these stained surfaces. Accuracy, in this case, would be indicated by minimal differences between the measured cross-sectional surface areas throughout the scaffold unit and the dimensioned sectioned surface areas. The dimensioned surface areas were collected (e.g., using SolidWorks®) through the intersection of a plane and the scaffold unit at the given height.

In implementations, the bone scaffold structure may be additionally constrained in the Z-axis by way of wire pitch angle, scaffold height, and/or geometric rules regarding the number, direction, and/or exact thickness of each wire, perhaps as a function of height, radius from implant center, or some other mechanical or rheological constraint as generated by geometric derivation from the root hexagonal tessellation. In one implementation, the bone scaffold structure may be engineered for selective permeability to certain types of bone cells by controlling the scaffold unit generation through throat distance, maximum clearance diameter, and the ratio of scaffold height to wire pitch angle to alter the formation of pores on the implant's cylindrical cage structure.

In some implementations, the bone scaffold structure is primarily derived from input control parameters used to tune the wires to some effect. For example, in certain implementations, a patient's bodyweight can be used as an input force against which the thickness of the wires in the scaffold structure may be optimized to minimize harmful stress shielding, a condition which prevents bone regrowth. A heavier patient would require a scaffold structure with thicker wires to attain the same stress deformation profile during, for instance, ambulation with a femoral implant. Further mechanical optimizations may largely pertain to, but are not limited to, the adjustment of the ratio of pores to material in a selected direction depending on the patient's native bone fluid flow rate and other osseous properties as deemed clinically relevant at the time of the scaffold structure's design and manufacture.

Implementations may have the advantages including classical open-reduction, external fixation (OREF) and most classical open-reduction, internal fixation (ORIF) techniques require secondary removal surgeries to prevent long-term side effects from implants. This implant does not have long-term side effects and can either remain in place or be resorbed depending on the material selected, reducing the number of surgeries to 1. Additionally, this bone scaffold structure design occupies less intramedullary space than current ORIF fixators, can be installed with less force, and can have slots designated for fracture fragments for easier surgical repair of a comminuted (shatter) fracture. This device uses the same sterile, proven surgical procedures as ORIF.

The bone scaffold structure as described may be used for long bone fracture repair for trauma reconstruction, bone regeneration for large segmental defects, and guided bone regrowth and regeneration, long term bone marrow drug dispenser (dispenses drug over degradation pathway), and mandibular and dental reconstruction.

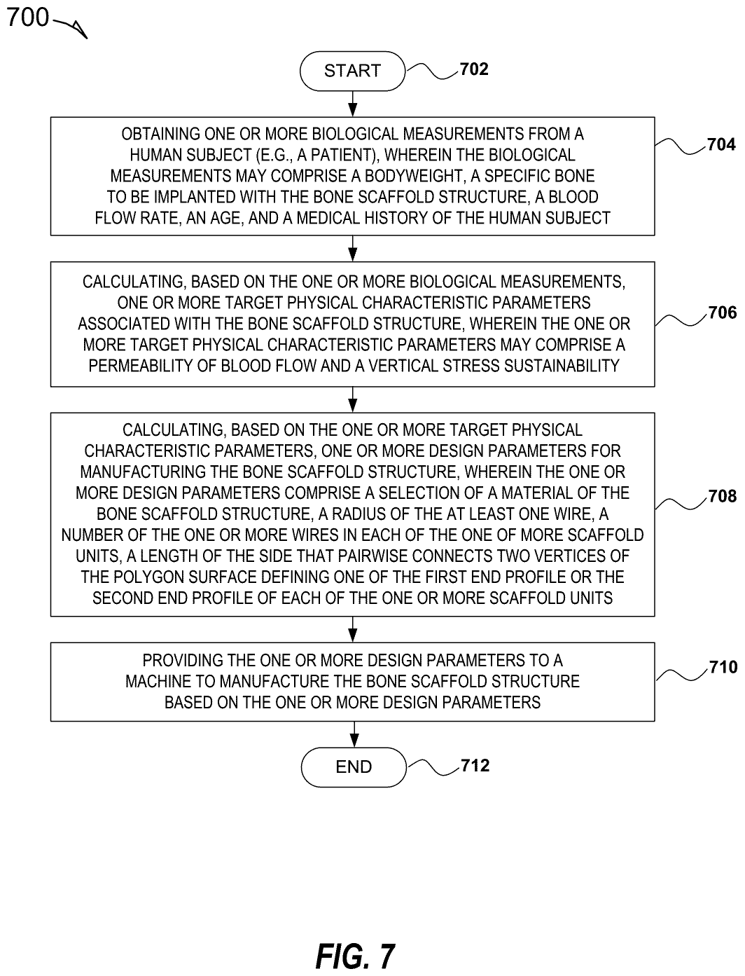

FIG. 7 shows a flowchart for a method for manufacturing a bone scaffold structure, according to some implementations of the present disclosure. A computer-implemented method 700 for designing a bone scaffold structure may begin at operation 702 and then, at operation 704, the method may include obtaining one or more biological measurements from a human subject (e.g., a patient), wherein the biological measurements may comprise a bodyweight, a specific bone to be implanted with the bone scaffold structure, a fluid flow rate, an age, and a medical history of the human subject. Other biological measurements may also be used if they may be useful for determining target physical characteristic parameters as described with respect to the next operation of method 700.

At operation 706 the method may include calculating, based on the one or more biological measurements, one or more target physical characteristic parameters associated with the bone scaffold structure, wherein the one or more target physical characteristic parameters may comprise a permeability to fluid flow and a vertical stress sustainability.

At operation 708 the method may include calculating, based on the one or more target physical characteristic parameters, one or more design parameters for manufacturing the bone scaffold structure, wherein the one or more design parameters comprise a selection of a material of the bone scaffold structure, a radius of the at least one wire, a number of the one or more wires in each of the one of more scaffold units, a length of the side that pairwise connects two vertices of the polygon surface defining one of the first end profile or the second end profile of each of the one or more scaffold units.

At operation 710 the method may include providing the one or more design parameters to a machine to manufacture the bone scaffold structure based on the one or more design parameters. As noted above, the tessellation of identical hexagon surfaces may be used in order to design an integral bone scaffold structure by using a software application (e.g., SolidWorks®) to copy the identical bone scaffold units defined by the hexagon surface as many times as needed. For example, the software application may use a fractal expansion algorithm to save modeling time by first designing the scaffold unit and then copying the scaffold unit around itself in a tessellation pattern until the pattern is sufficiently large for the desired profile geometry of the bone scaffold structure (e.g., a sufficient number of bone scaffold units have been produced).

At operation 712 the method 700 may end, for example, based on having manufactured the necessary number of scaffold units.

In some implementations, the outer boundaries of the bone scaffold structure may be trimmed based on the one or more design parameters for manufacturing the bone scaffold structure. In some implementations the scaffold structure may be further modified with surface modifications to enhance, for example, osteo-conductivity, osseointegration, and/or wound healing rate. In some implementations, the scaffold may be manufactured from a suitable resorbable biopolymer, including but not limited to polyethylene glycol (PETG). In other implementations, the scaffold may be made of a pure bioinert metal, metalloid, or alloy, provided that any alloys used have been confirmed to not harmfully leach alloying ions into surrounding tissue on abrasion, degradation, erosion, or any other form of wear. Bone scaffold structures may often need to be designed and manufactured in advance, while these scaffolds can be adjusted in software and 3D printed at the point of care. This may reduce inventory, shipping, and packaging costs while providing a quick and custom fit for a patient.

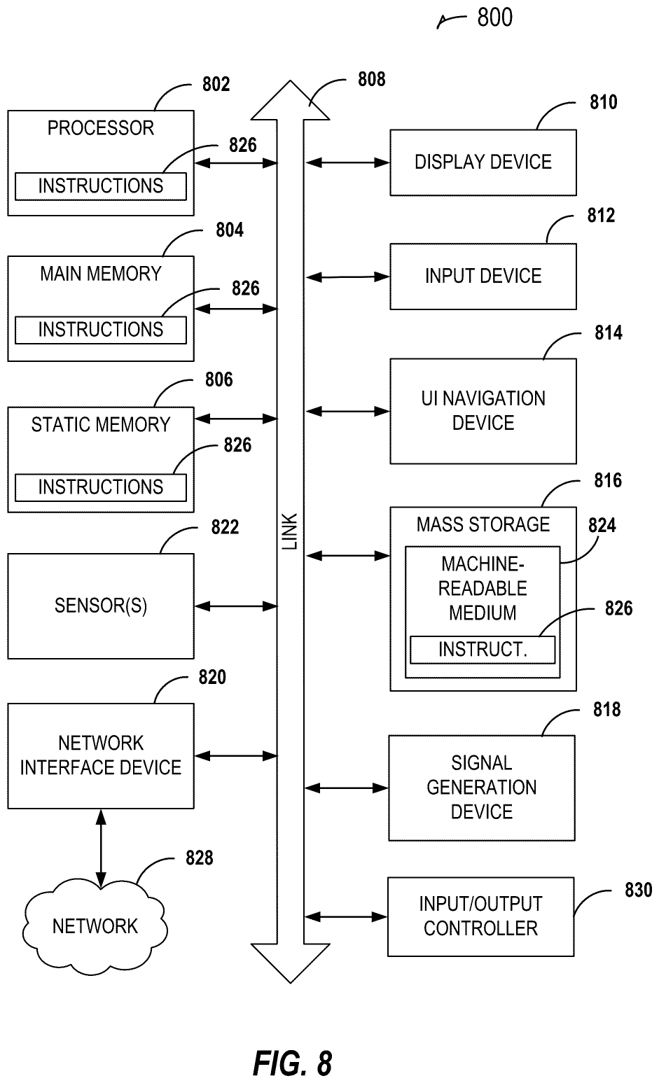

FIG. 8 is a block diagram illustrating a machine in the example form of a computer system 800, within which a set or sequence of instructions may be executed to cause the machine to perform any one of the methodologies discussed herein, according to some implementations of the present disclosure.

In alternative implementations, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be an onboard vehicle system, wearable device, personal computer (PC), a tablet PC, a hybrid tablet, a personal digital assistant (PDA), a mobile telephone, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein (e.g., method 700 of FIG. 7).

Example computer system 800 includes at least one processor 802 (e.g., a central processing unit (CPU), a graphics processing unit (GPU) or both, processor cores, compute nodes, etc.), a main memory 804 and a static memory 806, which communicate with each other via a link 808 (e.g., bus). The computer system 800 may further include a video display unit 810, an alphanumeric input device 812 (e.g., a keyboard), and a user interface (UI) navigation device 814 (e.g., a mouse). In one implementation, the video display unit 810, input device 812 and UI navigation device 814 are incorporated into a touch screen display. The computer system 800 may additionally include a storage device 816 (e.g., a drive unit), a signal generation device 618 (e.g., a speaker), a network interface device 820, and one or more sensors 822, such as a global positioning system (GPS) sensor, accelerometer, gyrometer, magnetometer, or other sensor.

The storage device 816 includes a machine-readable medium 824 on which is stored one or more sets of data structures and instructions 826 (e.g., software) embodying or utilized by any one or more of the methodologies or functions described herein. The instructions 826 may also reside, completely or at least partially, within the main memory 804, static memory 806, and/or within the processor 802 during execution thereof by the computer system 800, with main memory 804, static memory 806, and the processor 802 comprising machine-readable media.

While the machine-readable medium 824 is illustrated in an example implementation to be a single medium, the term “machine-readable medium” may include a single medium or multiple media (e.g., a centralized or distributed database, and/or associated caches and servers) that store the one or more instructions 826. The term “machine-readable medium” shall also be taken to include any tangible medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include volatile or non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 826 may further be transmitted or received over a

communications network 828 using a transmission medium via the network interface device 820 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 16G LTE/LTE-A or WiMAX networks). The term “transmission medium” shall be taken to include any intangible medium that is capable of storing, encoding, or carrying instructions for execution by the machine, and includes digital or analog signals or other intangible medium to facilitate communication of such software.

Example computer system 800 may also include an input/output controller 830 to receive input and output requests from at least one central processor 802, and then send device-specific control signals to the device they control. The input/output controller 830 may free at least one central processor 802 from having to deal with controlling each separate kind of device.

Language: In the foregoing description, numerous details are set forth. It will be apparent, however, to one of ordinary skill in the art having the benefit of this disclosure, that the present disclosure may be practiced without these specific details. In some instances, well-known structures and devices are shown in block diagram form, rather than in detail, in order to avoid obscuring the present disclosure.

Some portions of the detailed description have been presented in terms of algorithms generally conceived to be self-consistent sequences of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

Unless specifically stated otherwise as apparent from the following discussion, it is appreciated that throughout the description, discussions utilizing terms such as “segmenting”, “analyzing”, “determining”, “enabling”, “identifying” “modifying”, “parsing” or the like, refer to the actions and processes of a computer system, or similar electronic computing device, that manipulates and transforms data represented as physical (e.g., electronic) quantities within the computer system's registers and memories into other data represented as physical quantities within the computer system memories or other storage, transmission or display device.

The words “example” or “exemplary” are used herein to mean serving as an example, instance, or illustration. Any aspect or design described herein as “example’ or “exemplary” is not necessarily to be construed as preferred or advantageous over other aspects or designs. Rather, use of the words “example” or “exemplary” is intended to present concepts in a concrete fashion. Moreover, use of the term “an implementation” or “one implementation” or “an implementation” or “one implementation” throughout the disclosure is not intended to mean the same implementation or implementation unless described as such.

It is to be understood that the above description is intended to be illustrative, and not restrictive. Many other implementations/implementations will be apparent to those of skill in the art upon reading and understanding the above description.

The invention claimed is:

1. A bone scaffold structure, comprising:
one or more scaffold units, wherein each of the one or more scaffold units comprises a first end profile, a second end profile, and one or more wires connecting the first end profile to the second end profile,
wherein each of the first end profile and the second end profile is defined by a polygon surface that is specified by a center and a number (N) of vertices that are pairwise connected by a side, and
wherein the one or more wires are each specified by a wire length and a substantially circular cross-section, and wherein a first end of each of the one or more wires is connected to a vertex of the polygon surface defining the first end profile and a second end of each of the one or more wires is connected to a vertex of the polygon surface defining the second end profile.

2. The bone scaffold structure of claim 1, wherein the substantially circular cross-section of the at least one wire is defined by a center and a radius, and wherein the radius is shorter than a length of the side between any two adjacent vertices of the polygon surface of each of the one or more scaffold units.

3. The bone scaffold structure of claim 1, wherein the polygon surface that defines each of the first end profile and the second end profile of the one or more scaffold units comprises a hexagon surface that is specified by a center and six (6) vertices being pairwise connected by six (6) equal-length sides, and wherein a first end of each of the one or more wires is connected to a vertex of the hexagon surface that defines the first end profile, and a second end of each of the one or more wires is connected to a vertex of the hexagon surface that defines the second end profile.

4. The bone scaffold structure of claim 2, further comprising:
a first profile comprising a first tessellation of first end profiles of the one or more scaffold units, wherein the first tessellation comprises an integral arrangement of the hexagon surfaces of the first end profiles without gaps or overlapping; and
a second profile comprising a second tessellation of the second end profiles of the one or more scaffold units, wherein the second tessellation comprises an integral arrangement of the hexagon surfaces of the second end profiles without gaps or overlapping.

5. The bone scaffold structure of claim 3, wherein the one or more wires connecting the first end profile of each scaffold unit to the second end profile of the scaffold unit form a concentric pattern around a central axis of the scaffold unit with the first end of each wire being connected to a vertex of the hexagon surface that defines the first end profile at specified points located radially around the first end profile and the second end of each wire being connected to a corresponding vertex of the hexagon surface that defines the second end profile at corresponding points located radially around the second end profile.

6. The bone scaffold structure of claim 4, wherein each of the first profile and the second profile is specified according to a center and a plurality of sides, and wherein the center and the plurality of sides of the first profile are aligned with the center and the plurality of sides of the second profile resulting in a substantially zero degree pitch angle, for the wires connecting the first end profiles to the second end profiles, with respect to an axis connecting the center of the first profile and the center of the second profile.

7. The bone scaffold structure of claim 4, wherein each of the first profile and the second profile is specified according to a center and a plurality of sides, and wherein the center of the first profile in a scaffold unit is aligned with the center of the second profile, and the plurality of sides of the first profile is rotated at a parallel rotation angle with respect to a corresponding plurality of sides of the second profile resulting in a non-zero degree pitch angle, for the wires connecting the first end profiles to the second end profiles, with respect to an axis connecting the center of the first profile and the center of the second profile, and wherein the parallel rotation angle is more than zero degrees.

8. The bone scaffold structure of claim 7, wherein the wires connecting at least one of the first end profiles to a second end profile are counter-rotated around a center of the hexagon surface of the at least one first end profile to form a cage structure with cross-links and diamond-shaped pores between the at least one first end profile and the connected second end profile.

9. The bone scaffold structure of claim 8, further comprising a hole in the center of the hexagon surface of the at least one first end profile wherein the hole cuts through the full-thickness of the at least one first end profile and a corresponding hole in the center of the hexagon surface of the connected second end profile wherein the corresponding hole cuts through the full-thickness of the connected second end profile.

10. The bone scaffold structure of claim 9, wherein the specified points located radially around the first end profile and the corresponding points located radially around the second end profile are located at every 30 degrees of radial distance or 60 degrees of radial distance.

11. A computer-implemented method for designing and manufacturing the bone scaffold structure of claim 1, the method comprising:
obtaining one or more biological measurements from a human subject, wherein the biological measurements comprise at least one of a bodyweight, a bone to be implanted with the bone scaffold structure, a fluid flow rate, an age, or a medical history of the human subject;
calculating, based on the one or more biological measurements, one or more target physical characteristic parameters associated with the bone scaffold structure, wherein the one or more target physical characteristic parameters comprise at least one of a permeability to fluid flow or a vertical stress sustainability;
calculating, based on the one or more target physical characteristic parameters, one or more design parameters for manufacturing the bone scaffold structure, wherein the one or more design parameters comprise a selection of a material of the bone scaffold structure, a radius of the one or more wires, a number of the one or more wires in each of the one of more scaffold units, a length of the side that pairwise connects two vertices of the polygon surface defining one of the first end profile or the second end profile of each of the one or more scaffold units; and
providing the one or more design parameters to a machine to manufacture the bone scaffold structure based on the one or more design parameters.

12. The method of claim 11, wherein the machine comprises a 3D printer and the polygon surface comprises a hexagon surface.

13. The method of claim 12, wherein the one or more design parameters comprise a number of scaffold units required to manufacture the bone scaffold structure and the method further comprising using a fractal expansion algorithm to copy each of the first end profile and the second end profile around themselves in respective first and second tessellation patterns without gaps or overlapping until the first tessellation pattern includes a number of first end profiles equal to the number of scaffold units required to manufacture the bone scaffold structure and the second tessellation patterns includes a number of second end profiles equal to the number of scaffold units required to manufacture the bone scaffold structure.

14. The method of claim 13, further comprising trimming the outer boundaries of the bone scaffold structure based on the one or more design parameters for manufacturing the bone scaffold structure.

15. The method of claim 11, further comprising manufacturing the bone scaffold structure of resorbable biopolymer or of a pure bio-inert metal, metalloid, or alloy.

16. A bone scaffold structure, comprising:
a first profile, a second profile, and a plurality of wires connecting from a plurality of connection points on the first profile to a plurality of connection points on the second profile, wherein the plurality of connection points on the first profile are located at vertices of polygons associated with a first tessellation of polygons on a surface of the first profile, and the plurality of connection points on the second profile are located at vertices of polygons associated with a second tessellation of polygons on a surface of the second profile.

17. The bone scaffold structure of claim 16, wherein the first profile and the second profile are identical in shape, and wherein the first profile and the second profile are one of aligned with each other or rotated at a non-zero degree in the range of (0, 360) degrees with respect to an axis connecting a center of the first profile and a center of the second profile.

18. The bone scaffold structure of claim 16, wherein each of the first tessellation of polygons and the second tessellation of polygons comprises a tessellation of identical hexagons, and wherein the plurality of connection points on the first profile are located at vertices of hexagons associated with a first tessellation of hexagons on a surface of the first profile, and the plurality of connection points on the second profile are located at vertices of hexagons associated with a second tessellation of hexagons on a surface of the second profile.

19. The bone scaffold structure of claim 17, wherein the hexagons are adjacently arranged so that each hexagon is specified by a center, six sides, and six vertices, and each hexagon shares a common side and two vertices with a neighboring hexagon, and wherein the adjacently arranged hexagons are tightly connected but do not overlap with each other.

20. The bone scaffold structure of claim 17, wherein each vertex of each hexagon in the first and second tessellations of hexagons is connected to a corresponding wire in the plurality of wires, and wherein each wire in the plurality of wires has a circular cross-section and a length.
